(19) United States
(12) Patent Application Publication
(10) Pub. No.: US 2020/0121056 A1
(43) Pub. Date: Apr. 23, 2020

(54) METHOD, SYSTEM AND COMPOSITION FOR SUNLESS TANNING

(71) Applicant: Helios Global, LLC, Austin, TX (US)

(72) Inventor: Patricia Huddleston, Austin, TX (US)

(21) Appl. No.: 16/163,173

(22) Filed: Oct. 17, 2018

(51) Int. Cl. A45D 34/04; A61K 8/35; A61Q 19/04; A61K 8/9794; A61K 8/36; A61K 8/73; A61K 8/34; A45D 33/00

(52) U.S. Cl. CPC A45D 34/041; A61K 8/35; A61Q 19/04; A61K 8/9794; A61K 8/36; A46B 9/021; A61K 8/345; A45D 33/00; A61K 2800/874; A61K 2800/42; A45D 2200/25; A61K 8/735

(57) ABSTRACT

A method and system for sunless tanning includes a rolling applicator system having an applicator frame joined to a handle, a first roller head mounting axle, a first roller head applicator supported for rotation relative to the applicator frame for application of sunless tanning liquid composition to the skin, and a second roller head applicator supported for redistribution of excess sunless tanning liquid composition on the skin to provide a uniform visual appearance.

### CROSS REFERENCE TO RELATED APPLICATIONS

This application at the date of filing is related to no other applications.

### FIELD OF THE INVENTION

The disclosure relates generally to methods, systems, apparatus and compositions for sunless tanning.

### BACKGROUND OF THE INVENTION

Methods, systems and compositions for sunless tanning may be understood by categorizing the modes of application. One category or mode is manual application of a preformulated sunless tanning lotion, by rubbing the lotion on the skin. A second category is manual application of a preformulated sunless tanning aerosol spray, from a prepackaged spray can receptacle. Both the first and second categories or modes of sunless tanning may be performed by the individual end-user receiving the sunless tan, or by a second person assisting the end-user. Visual results achieved by the first and second categories of sunless tanning typically are lacking in significant ways. Unnaturally thick and non-uniform coverage by lotion or formula, and variations in coloring between areas of the skin, may be readily visible. Sunless tans of the first and second categories may not provide an appearance approximating either a natural tan achieved by sunning the body outdoors under natural sunlight, or under the artificial sunlight of a tanning bed. Sunless tans of the first and second categories may exhibit clumping of lotion in undesired agglomerations or lumps on the skin, undesired and unnatural coloring biased towards orange shades or “oranging out”; and propensity for the sunless tan to be removed from the skin in spots when the end-user comes into contact with objects in the surrounding environment.

A third category or mode of sunless tanning is spray application of a sunless tanning solution by a trained technician using a sprayer driven by an air compressor, followed by active drying in front of a fan. Compressor-driven sprayers of the sort used by professional painters to apply paint to surfaces of buildings may be suitable. Application of spray tans by a technician using a compressor-driven sprayer typically provides better visual results than achieved by the first and second categories or modes of sunless tanning. Usage of compressor-driven spray tanning equipment, however, is lacking in significant ways. The technician may be required to tune the sprayer to compensate for factors such as variations in temperature and viscosity of the sunless tanning solution. The technician must be trained and experienced using the equipment. The solution may run on the skin when thin, or may be may have unnatural and non-uniform coverage and appearance where the end-user changes positions and fails to maintain a consistent posture during the application and drying under fan for an extended period. It is disadvantageous, however, that application in this manner may occupy significant space and require extensive use of plastic sheet barriers to limit overspray, extensive efforts for cleaning up overspray, or both. The technician must be trained to use the compressor-driven sprayer, which is complex and may be hazardous to the end-user where a technician is untrained or poorly trained. Spray tanning may require the end-user and technician to wear protective respiratory equipment to ensure safety. Spray tanning may require the technician to perform repeated cleaning cycles to protect the spray equipment from damage. Spray tanning solution may be unusable after being dispensed into such a sprayer. Spray tanning may require the end-user to stand in front of a fan for an extended period, in order to dry the solution on the end-user's skin. Use of a fan may redistribute solution on the skin in undesired patterns. Changes in posture during the application and fan drying periods may damage uniformity, coverage and appearance of the sunless spray tan. Operating compressor-driven spray tanning equipment may produce significant, undesired noise. A compressor driven spray tanning unit may not be readily portable for use in areas such as a residence, and may require a dedicated, fixed location.

### BRIEF DESCRIPTION OF THE INVENTION

The above-mentioned shortcomings, disadvantages and problems are addressed herein, as will be understood by those skilled in the art upon reading and studying this specification including the accompanying drawings. This brief description is a summary provided to introduce a selection of concepts in a simplified form, that are further described below in more detail in the Detailed Description following. This brief description is not intended to identify key or essential features of the claimed subject matter. Unless otherwise defined, all terms (including technical and scientific terms) used herein have the same meaning as commonly understood by one of ordinary skill in the field of art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

In one aspect, a system for sunless tanning may include a rolling applicator system, sunless tanning liquid composition, and a loading receptacle. The rolling applicator system may include an applicator frame for manual movement. The applicator frame may include a roller head mounting axle configured to support a roller head applicator for rotation relative to the applicator frame. The roller head applicator is supported for rotation in relation to the applicator frame by the roller head mounting axle. The roller head applicator may include a roller cover including fluid transfer media disposed for rolling movement upon rotation of the roller head applicator. The fluid transfer media may by configured to transfer sunless tanning liquid composition during such rolling movement. The sunless tanning liquid composition may include 1,3-dihydroxyacetone (C₃H₆O₃). A liquid transfer receptacle may be configured to contain sunless tanning liquid composition in direct fluid communication with said fluid transfer media to load the fluid transfer media with said sunless tanning liquid composition. The fluid transfer media may comprise a nap roller cover including a nap formed of pile fabric for unloading sunless tanning liquid composition to the skin. The fluid transfer media may comprise a roller cover formed of foam for taking up and redistributing excess of the sunless tanning liquid composition on the skin to provide uniform, natural visual appearance.

Apparatus, systems, and methods of varying scope are described herein. These aspects are indicative of various non-limiting ways in which the disclosed subject matter may be utilized, all of which are intended to be within the scope of the disclosed subject matter. In addition to the aspects and advantages described in this summary, further aspects, features, and advantages will become apparent by reference to the associated drawings, detailed description, and claims.

### BRIEF DESCRIPTION OF THE DRAWINGS

The disclosed subject matter itself, as well as further objectives, and advantages thereof, will best be illustrated by reference to the following detailed description of embodiments of the device read in conjunction with the accompanying drawings, wherein:

FIG. 1 is a simplified flowchart illustrating a method for sunless tanning in an exemplary embodiment.

FIG. 2 is a simplified schematic diagram illustrating a system for sunless tanning in an exemplary embodiment.

FIG. 3 is a simplified schematic diagram illustrating a system for sunless tanning in an exemplary embodiment.

### DETAILED DESCRIPTION OF THE INVENTION

In the following detailed description, reference is made to the accompanying drawings which form a part hereof, and in which is shown by way of illustration specific embodiments which may be practiced. These embodiments are described in sufficient detail to enable those skilled in the art to practice the embodiments and disclosure. It is to be understood that other embodiments may be utilized, and that logical, mechanical, electrical, and other changes may be made without departing from the scope of the embodiments and disclosure. In view of the foregoing, the following detailed description is not to be taken as limiting the scope of the embodiments or disclosure.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be appreciated that for simplicity and clarity of illustration, where considered appropriate, reference numerals may be repeated among the figures to indicate corresponding or analogous elements. In addition, numerous specific details are set forth in order to provide a thorough understanding of the implementations described herein. However, it will be understood by those of ordinary skill in the art that the implementations described herein may be practiced without these specific details. In other instances, well-known methods, procedures and components have not been described in detail so as not to obscure the implementations described herein. Also, the description is not to be considered as limiting the scope of the implementations described herein.

The detailed description set forth herein in connection with the appended drawings is intended as a description of exemplary embodiments in which the presently disclosed apparatus and system can be practiced. The term “exemplary” used throughout this description means “serving as an example, instance, or illustration,” and should not necessarily be construed as preferred or advantageous over other embodiments.

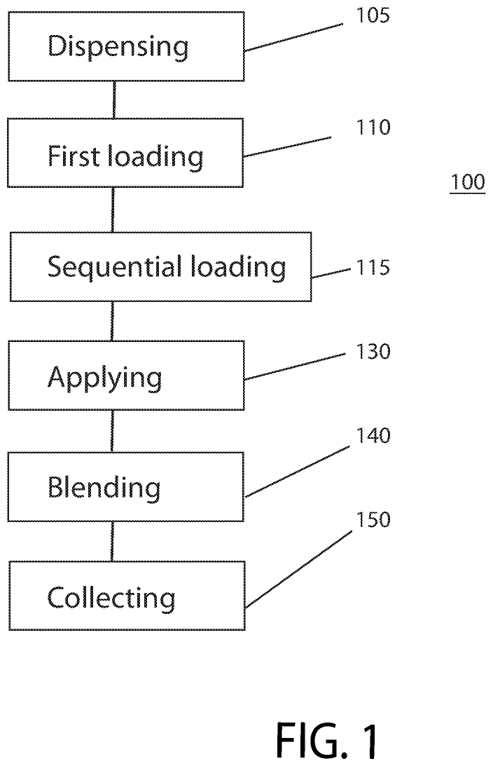

Illustrated in FIG. 1 is a simplified flowchart illustrating a method 100 for sunless tanning in an exemplary embodiment. Referring to FIG. 1, method 100 may include dispensing 105 into a liquid transfer receptacle a volume of sunless tanning liquid composition comprising 1,3-dihydroxyacetone (C₃H₆O₃). In such dispensing 105, the liquid transfer receptacle may be configured to contain the sunless tanning liquid composition with a full-width section of a roller cover of a roller head applicator partially immersed in the sunless tanning liquid composition.

Referring to FIG. 1, method 100 may include first loading 110 the sunless tanning liquid composition on fluid transfer media of a first full-width section of a roller cover of the first roller head applicator. First loading 110 may include partial immersion to establish direct fluid communication of the sunless tanning liquid composition with the fluid transfer media of the first full-width section.

Referring to FIG. 1, method 100 may include sequential loading 115, in circumferential progression, the sunless tanning liquid composition on the fluid transfer media of each other full-width section of the roller cover. Sequential loading 115 may include rolling movement of the roller head applicator in partial immersion in the sunless tanning liquid composition to establish direct fluid communication of the sunless tanning liquid composition with the fluid transfer media of each other full-width section in circumferential progression around the roller cover.

Referring to FIG. 1, method 100 may include applying 130, by rolling movement of the roller head applicator, the sunless tanning liquid composition to the skin. In such applying 130, the roller head applicator may include a nap roller cover including a nap formed of pile fabric.

Referring to FIG. 1, method 100 may include blending 140, by rolling movement of a second roller head applicator comprising a foam roller cover comprising foam, the sunless tanning liquid composition on skin of a subject individual.

Referring to FIG. 1, method 100 may include: collecting 150, by application of a powder mixture to the skin with a powder applicator, excess amounts of the sunless tanning liquid composition from the skin.

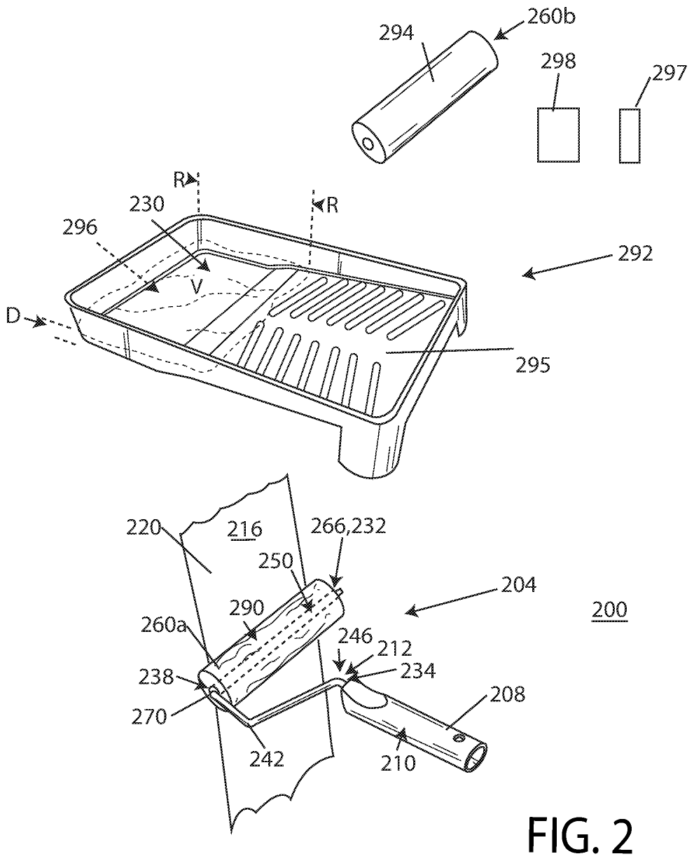

FIG. 2 is a simplified block diagram illustrating a system 200 for sunless tanning in an exemplary embodiment. System 200 for sunless tanning may include a rolling applicator system 204. Rolling applicator system 204 may include a handle 208 including a grip area 210 for manual gripping by a user. Handle 208 may be configured to support an applicator frame 212 for manual movement, such as translation movement, relative to skin 216 of the body of an end-user or subject to dispense and apply sunless tanning liquid composition 230 from rolling applicator system 204 to skin 216 of the subject.

As shown in FIG. 2, applicator frame 212 may be joined to handle 208. Applicator frame 212 may include a proximal end 234 adjoining handle 208. Applicator frame 212 may include a distal end 238 spaced from handle 208 and adjoining proximal end 234 in spaced relation to handle 208, with proximal end 234 located intermediate handle 208 and distal end 238. In some embodiments, applicator frame 212 may include an elongated rod frame member 242 shaped to define applicator frame 212. Rod frame member 242 may include neck 246 joined to handle 208. Rod member 242 may extend away from neck 246 to define a roller head axle 250 opposite handle 208. Roller head axle 250 may include a spaced pair of roller head mounts (not shown) located at opposite ends thereof. The roller head mounts may be configured to support a roller head applicator 260a for rotation relative to applicator frame 212 about a roller head mounting axis defined between the spaced pair of roller head mounts and extending along roller head axle 250. It will be understood that, in the particular embodiment shown in FIG. 2, the roller head mounts are portions of roller head axle 250.

Referring to FIG. 2, roller head applicator 260a may include a roller head body 270 having a roller head hub defining an open roller head aperture. Roller head applicator 260a may have a roller head major axis extending through roller head aperture. The roller head axle may extend through the open roller head aperture when roller head applicator 260a is installed and mounted on roller head axle 250. Roller head body 270 may be supported for rotation in common with the roller head hub about the roller head axle 250, when roller head hub is supported on the roller head mounts on roller head axle 250. It will be understood that, in some embodiments, when roller head applicator 260a is mounted on roller head axle 250, the roller head major axis may be coextensive with roller head mounting axis extending along the roller head axle 250 of applicator frame 212.

Referring to FIG. 2, roller head applicator 260a may include a roller cover 266 supported on roller head body 270 in spaced radial relationship to the roller head hub. Roller cover 266 may include fluid transfer media 290 disposed for rolling movement across the skin 216 upon rotation of the roller head applicator 260a on roller head axle 250. The fluid transfer media 290 may cover all surface area of the roller cover 266. The fluid transfer media 290 may be configured to transfer sunless tanning liquid composition 230 during rolling movement across the skin 216. The sunless tanning liquid composition 230 may include 1,3-dihydroxyacetone (C₃H₆O₃), as elsewhere described herein.

Referring to FIG. 2, system 200 may include a liquid transfer receptacle 292. Liquid transfer receptacle 292 may be configured to contain the sunless tanning liquid composition 230 in direct fluid communication with the fluid transfer media 290 for loading the fluid transfer media 290 with sunless tanning liquid composition 230 when the roller cover 266 is dipped (not shown in FIG. 2) into the sunless tanning liquid composition 230 in liquid transfer receptacle 292. Referring to FIG. 2, The fluid transfer media 290 may be configured to dispense and apply the sunless tanning liquid composition 230 to the skin 216 of the end-user subject 220, for darkening the visual appearance of such skin 216. Referring to FIG. 2, liquid transfer receptacle 292 may include a roller tray pan 295 configured to enable loading of the fluid transfer media 290 covering said all surface area of the nap roller cover 232, by rolling movement of the nap roller cover 266 across the roller tray pan 295 to coat the fluid transfer media 290 with said sunless tanning liquid composition 230. In some embodiments, roller tray pan 295 may be configured to enable loading of the fluid transfer media 290 covering all surface area of the nap roller cover 266 by rolling movement of the nap roller cover 266 across the roller tray pan 295 in a predetermined volume (V) of the sunless tanning liquid composition 230. The predetermined volume may be controlled to conserve sunless tanning liquid composition 230 by filling the roller tray pan 295 with the sunless tanning liquid composition 230 to a selected initial depth (D) sufficient for loading the fluid transfer media 290 covering said surface area of the roller cover 266. In some embodiments, roller pan tray 295 may have a bottom surface 296 defining a roller runway (RR) having a runway length exceeding circumference of the nap roller cover 266 to enable one revolution of rolling movement of the nap roller cover 266 across the roller runway (RR) partially immersed in the predetermined minimum volume of sunless tanning liquid composition 230.

In the particular embodiment shown in FIG. 2, the fluid transfer media 290 may include a nap roller cover 232 installed and supported on, and extending around, the roller head body 270 of roller head applicator 260a. Such a nap roller cover 232 may include a pile fabric. Nap roller cover 232 including pile fabric may be configured to take on or load the sunless tanning liquid composition 230 from liquid transfer receptacle 292, and also to dispense or unload the sunless tanning liquid composition 230 to the skin 216.

As shown in FIG. 2, system 200 may include second roller head applicator 260b that is a foam roller 294. In the particular embodiment shown in FIG. 2, the second roller head applicator 260b is interchangeable with roller head applicator 260a having nap roller cover 232, to be mounted on roller head mounting axle 250 and removable therefrom. Such foam roller 294 may include a foam roller cover including foam. It will be understood that such foam may be configured to take up excess or uneven accumulations of sunless tanning liquid composition 230 from the skin 216, and to carry and redistribute sunless tanning liquid composition 230 to provide a uniform visual appearance on the skin 216. Foam roller 294 may be configured to selectively release or unload sunless tanning liquid composition 230. It will be understood that foam roller 294 conserves sunless tanning liquid composition 230 by taking up and redistributing excess or uneven accumulations and thus reducing need to dispense additional of the sunless tanning liquid composition 230.

Referring to FIG. 2, system 200 for sunless tanning may include the sunless tanning liquid composition 230 having a major portion of liquid base material. In some embodiments, the liquid base material may be selected from the following: water; aloe vera juice; and mixtures of the preceding.

Sunless tanning liquid composition 230 may include a color complexity agent. In an embodiment, such color complexity agent may include, or may consist of, erythrulose (C₄H₈O₄).

In some embodiments, sunless tanning liquid composition 230 may include a drying agent. In embodiments, the drying agent may include an alcohol.

In some embodiments, the sunless tanning liquid composition 230 of system 200 may include a moisturizer agent. In some embodiments, the moisturizer agent may include hyaluronic acid.

System 200 for sunless tanning may include the sunless tanning liquid composition 230 including a stabilizer agent. In some embodiments, the stabilizer agent may include, or may consist of, propyl glycol.

Referring to FIG. 2, system 200 may include a powder applicator 297 configured to apply a powder mixture 298 to collect excess of the sunless tanning liquid composition from skin 216. In an embodiment, for example, a powder mixture 298 may include arrowroot powder. In some embodiments, the powder applicator 297 may include a brush configured to apply the powder mixture 298.

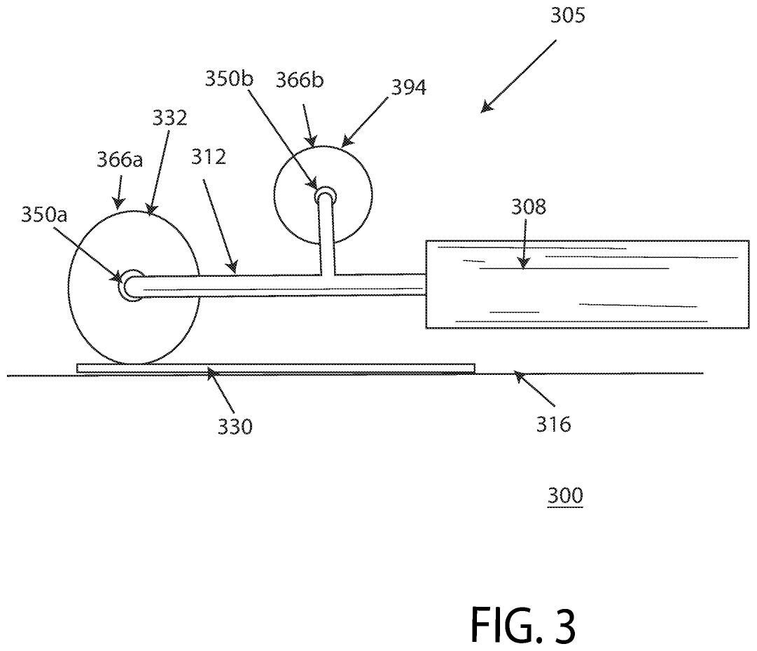

Referring to FIG. 3, a system 300 for sunless tanning may include a rolling applicator system 305. Except as otherwise described here or illustrated in FIG. 3, rolling applicator system 305 may be identical to rolling applicator system 204 illustrated in preceding FIG. 2 and described elsewhere herein. Rolling applicator system 305 is configured to apply sunless tanning liquid composition. Rolling applicator system 305 includes applicator frame 312. Applicator frame 312 includes first roller head axle 350a and second roller head axle 350b. Rolling applicator system 305 includes first roller head applicator 366a mounted on first roller head axle 350a, and second roller head applicator 366b mounted on second roller head axle 350b. First roller head applicator 366a may include nap roller cover 332. Second roller head applicator 366b may include foam roller cover 394. One of the handle 308 and the applicator frame 312 may be configured to selectably locate the first roller head applicator 366a in an active position in contact with the skin 316 for application of sunless tanning liquid composition 330, with the second roller head applicator 366b in an inactive position. One of the handle 308 and the applicator frame 312 may be configured to selectably locate the second roller head applicator 366b in an active position in contact with the skin 316 for redistribution of sunless tanning liquid composition 330, with the first roller head applicator 366a in an inactive position.

Apparatus, methods and systems according to embodiments of the disclosure are described. Although specific embodiments are illustrated and described herein, it will be appreciated by those of ordinary skill in the art that any arrangement which is calculated to achieve the same purposes can be substituted for the specific embodiments shown. This application is intended to cover any adaptations or variations of the embodiments and disclosure. For example, although described in terminology and terms common to the field of art, exemplary embodiments, systems, methods and apparatus described herein, one of ordinary skill in the art will appreciate that implementations can be made for other fields of art, systems, apparatus or methods that provide the required functions. The scope of subject matter disclosed or claimed herein should therefore not be limited by the above described embodiment, method, and examples, but by all embodiments and methods within the scope and spirit of the invention. Although methodologies have been particularly described in the foregoing disclosure, it is to be understood that such descriptions have been provided for purposes of illustration only, and that other variations both in form and in detail can be made thereupon by those skilled in the art without departing from the spirit and scope thereof, which is defined first and foremost by the appended claims. Apparatus, methods and systems according to embodiments of the disclosure are described. Although specific embodiments are illustrated and described herein, it will be appreciated by those of ordinary skill in the art that any arrangement which is calculated to achieve the same purposes can be substituted for the specific embodiments shown. This application is intended to cover any adaptations or variations of the embodiments and disclosure. For example, although described in terminology and terms common to the field of art, exemplary embodiments, systems, methods and apparatus described herein, one of ordinary skill in the art will appreciate that implementations can be made for other fields of art, systems, apparatus or methods that provide the required functions. The invention should therefore not be limited by the above described embodiment, method, and examples, but by all embodiments and methods within the scope and spirit of the invention. In particular, one of ordinary skill in the art will readily appreciate that the names of the methods and apparatus are not intended to limit embodiments or the disclosure. Furthermore, additional methods, steps, and apparatus can be added to the components, functions can be rearranged among the components, and new components to correspond to future enhancements and physical devices used in embodiments can be introduced without departing from the scope of embodiments and the disclosure. One of skill in the art will readily recognize that embodiments are applicable to future systems, future apparatus, future methods, and different materials. All methods described herein can be performed in a suitable order unless otherwise indicated herein or otherwise clearly contradicted by context. The use of any and all examples, or exemplary language (e.g., “such as”), is intended merely to better illustrate the disclosure and does not pose a limitation on the scope of the disclosure unless otherwise claimed. No language in the specification should be construed as indicating any non-claimed element as essential to the practice of the disclosure as used herein. Terminology used in the present disclosure is intended to include all environments and alternate technologies that provide the same functionality described herein.

In particular, one of ordinary skill in the art will readily appreciate that the names of the methods and apparatus are not intended to limit embodiments or the disclosure. Furthermore, additional methods, steps, and apparatus can be added to the components, functions can be rearranged among the components, and new components to correspond to future enhancements and physical devices used in embodiments can be introduced without departing from the scope of embodiments and the disclosure. One of skill in the art will readily recognize that embodiments are applicable to future systems, future apparatus, future methods, and different materials.

All methods described herein can be performed in a suitable order unless otherwise indicated herein or otherwise clearly contradicted by context. The use of any and all examples, or exemplary language (e.g., “such as”), is intended merely to better illustrate the disclosure and does not pose a limitation on the scope of the disclosure unless otherwise claimed. No language in the specification should be construed as indicating any non-claimed element as essential to the practice of the disclosure as used herein.

Terminology used in the present disclosure is intended to include all environments and alternate technologies that provide the same functionality described herein.

1. A system for sunless tanning, said system comprising:
a rolling applicator system comprising:
a handle configured to support an applicator frame for manual movement, said handle comprising a grip area;
said applicator frame joined to said handle, said applicator frame comprising a roller head mounting axle spaced from said handle, said roller head mounting axle configured to support a roller head applicator for rotation relative to said applicator frame;
said roller head applicator supported for rotation in relation to said roller head mounting axle, said roller head applicator including a roller cover comprising fluid transfer media disposed for rolling movement upon rotation of said roller head, said fluid transfer media configured to transfer sunless tanning liquid composition during said rolling movement;
said sunless tanning liquid composition comprising 1,3-dihydroxyacetone (C₃H₆O₃); and
a liquid transfer receptacle configured to contain said sunless tanning liquid composition in direct fluid communication with said fluid transfer media to load said fluid transfer media with said sunless tanning liquid composition.

2. A system for sunless tanning according to claim 1, further comprising:
said fluid transfer media comprising a nap roller, said nap roller comprising a roller cover including a nap formed of pile fabric.

3. A system for sunless tanning according to claim 1, further comprising:
said fluid transfer media comprising a foam roller, said foam roller comprising a roller cover formed of foam.

4. A system for sunless tanning according to claim 1, further comprising:
said fluid transfer media covering all surface area of said roller cover;
said liquid transfer receptacle comprising a roller tray pan configured to enable loading of said fluid transfer media covering said all surface area by rolling movement of said roller cover across said roller tray pan to coat said fluid transfer media covering said all surface area with said sunless tanning liquid composition.

5. A system for sunless tanning according to claim 1, further comprising:
said fluid transfer media covering all surface area of said roller cover;
said liquid transfer receptacle comprising a roller tray pan configured to enable loading of said fluid transfer media covering said all surface area by rolling movement of said roller cover across said roller tray pan in a predetermined volume of said sunless tanning liquid composition, said predetermined volume controlled to conserve said sunless tanning liquid composition by filling said roller tray pan with said sunless tanning liquid composition to a selected initial depth sufficient for loading said fluid transfer media covering said all surface area.

6. A system for sunless tanning according to claim 1, further comprising:
said fluid transfer media covering all surface area of said roller cover;
said liquid transfer receptacle comprising a roller tray pan configured to enable loading of said fluid transfer media covering said all surface area by rolling movement of said roller cover across said roller tray pan in a predetermined volume of said sunless tanning liquid composition, said predetermined volume controlled to conserve said sunless tanning liquid composition by filling said roller tray pan with said sunless tanning liquid composition to a selected initial depth sufficient for loading said fluid transfer media covering said all surface area,
said roller pan tray having a bottom surface defining a roller runway having a runway length exceeding circumference of said roller cover to enable one revolution of rolling movement of said roller cover across said roller runway partially immersed in said predetermined minimum volume of said sunless tanning liquid composition.

7. A system for sunless tanning according to claim 1, further comprising:
said sunless tanning liquid composition comprising a major portion of liquid base material selected from the following:
water;
aloe vera juice; and
both of the preceding.

8. A system for sunless tanning according to claim 1, further comprising:
said sunless tanning liquid composition comprising a color complexity agent comprising erythrulose (C₄H₈O₄).

9. A system for sunless tanning according to claim 1, further comprising:
said sunless tanning liquid composition comprising a drying agent comprising an alcohol.

10. A system for sunless tanning according to claim 1, further comprising:
said sunless tanning liquid composition comprising a moisturizer agent comprising hyaluronic acid.

11. A system for sunless tanning according to claim 1, further comprising:
said sunless tanning liquid composition comprising a stabilizer agent comprising propyl glycol.

12. A system for sunless tanning according to claim 1, further comprising:
a powder applicator;
a powder mixture to collect excess of said sunless tanning liquid composition, said powder mixture comprising arrowroot powder.

13. A system for sunless tanning according to claim 12, further comprising:
said powder applicator comprising a brush.

14. A method for sunless tanning, said method comprising:
dispensing into a liquid transfer receptacle a volume of sunless tanning liquid composition comprising 1,3-dihydroxyacetone (C₃H₆O₃), said liquid transfer receptacle configured to contain said sunless tanning liquid composition with a full-width section of a roller cover of a roller head applicator partially immersed in said sunless tanning liquid composition;
first loading said sunless tanning liquid composition on fluid transfer media of a first full-width section of a roller cover of said roller head applicator, by partial immersion to establish direct fluid communication of said sunless tanning liquid composition with said fluid transfer media of said first full-width section;
sequential loading, in circumferential progression, said sunless tanning liquid composition on said fluid transfer media of each other full-width section of said roller cover, by rolling movement of said roller head applicator in partial immersion in said sunless tanning liquid composition to establish direct fluid communication of said sunless tanning liquid composition with said fluid transfer media of each other said first full-width section in circumferential progression around said roller cover.

15. A method for sunless tanning according to claim 14, said method comprising:
applying, by rolling movement of said roller head applicator, said sunless tanning liquid composition.

16. A method for sunless tanning according to claim 15, said method comprising:
in said applying, said roller head applicator comprising a nap of pile fabric unloading said sunless tanning liquid composition.

17. A method for sunless tanning according to claim 14, said method comprising:
blending, by rolling movement of a second roller head applicator comprising a second roller cover comprising foam taking up excess of said sunless tanning liquid composition from the skin.

18. A method for sunless tanning according to claim 14, said method comprising:
collecting, by application of a powder mixture by a powder applicator, excess of said sunless tanning liquid composition accumulated with said powder mixture.

19. A system for sunless tanning, said system comprising:
a rolling applicator system configured to apply sunless tanning liquid composition, said rolling applicator system comprising:
a handle configured to support an applicator frame for manual movement, said handle comprising a grip area;
said applicator frame joined to said handle, said applicator frame comprising a roller head mounting axle spaced from said handle, said roller head mounting axle configured to support a roller head applicator for rotation relative to said applicator frame about said roller head mounting axle;
said roller head applicator supported for rotation about said roller head mounting axle, said roller head applicator including a roller cover comprising fluid transfer media disposed for rolling movement upon rotation of said roller head, said fluid transfer media configured to transfer sunless tanning liquid composition during said rolling movement;
and
said sunless tanning liquid composition comprising 1,3-dihydroxyacetone (C₃H₆O₃).

20. A rolling applicator system configured to apply sunless tanning liquid composition, said rolling applicator system comprising:
a handle configured to support an applicator frame for manual movement, said handle comprising a grip area;
said applicator frame joined to said handle, said applicator frame comprising a first roller head mounting axle, said applicator frame comprising a second roller head mounting axle;
said first roller head mounting axle spaced from said handle, said first roller head mounting axle configured to support a first roller head applicator for rotation relative to said applicator frame about said first roller head mounting axle;
said second roller head mounting axle spaced from said handle, said second roller head mounting axle configured to support a second roller head applicator for rotation relative to said applicator frame about said second roller head mounting axle;
one of said handle and said applicator frame configured to selectably locate said first roller head applicator in an active position in contact with skin for application of sunless tanning liquid composition, with said second roller head applicator located in an inactive position; and
one of said handle and said applicator frame configured to selectably locate said second roller head applicator in an active position in contact with skin for redistribution of sunless tanning liquid composition to provide uniform visual appearance, with said first roller head applicator located in an inactive position.

21. A rolling applicator system according to claim 20, said rolling applicator system comprising:
said first roller head applicator comprising a nap formed of pile fabric,
said second roller head applicator comprising a foam cover.

22. A kit for sunless tanning, said kit comprising:
a system for sunless tanning according to claim 19.
